(19) United States
(12) Patent Application Publication
(10) Pub. No.: US 2020/0279472 A1
(43) Pub. Date: Sep. 3, 2020

(54) HAND WASHING MONITORING DEVICE, SYSTEM AND METHOD

(71) Applicant: Fian Technologies Inc., Toronto (CA)

(72) Inventors: Parham Chinikar, Toronto (CA); Marawan Abdel Hameed, Toronto (CA); Ming Jia Michael Wu, Toronto (CA); Luke Kyne, Toronto (CA)

(21) Appl. No.: 16/793,087

(22) Filed: Feb. 18, 2020

(30) Foreign Application Priority Data
Feb. 28, 2019 (CA) 3035191

(51) Int. Cl. G08B 21/24; E03C 1/05; G16H 40/20; A61B 5/11

(52) U.S. Cl. CPC G08B 21/245; E03C 1/057; G16H 40/20; A47K 5/1217; A61B 5/1126; A47K 2210/00; A61B 5/1123

(57) ABSTRACT

Described are various embodiments of a hand wash monitoring device, system and method. In some embodiments, the device is to be mounted in a vicinity of a corresponding sink to monitor, for a given user, a designated hand washing sequence of designated hand washing activities comprising at least a sink-based washing activity and an external washing activity in accordance with respective prescribed washing activity durations.

### CROSS REFERENCE TO RELATED APPLICATION

This application claims the benefit of priority to Canadian Patent Application No. 3,035,191, filed Feb. 28, 2019 and entitled “HAND WASHING MONITORING DEVICE, SYSTEM AND METHOD” which is incorporated by reference herein in its entirety.

### FIELD OF THE DISCLOSURE

The present disclosure relates generally to systems and methods for monitoring compliance with hand-washing practices, and, in particular, to a hand washing monitoring device, system and method.

### BACKGROUND

Infectious diseases, many of which are spread by unclean hands, remain a leading cause of death and disease worldwide. Washing hands with soap and clean water for 10-20 seconds is a sensible strategy for hand hygiene in healthcare-related or non-healthcare settings, and is recommended by the CDC and other experts. Unfortunately, users tend to wash their hands quickly without following the recommended guidelines. Therefore, it is sometime useful to have devices and/or systems in place to provide guidelines and/or to monitor users washing their hands to ensure that they do so properly.

Various hand hygiene monitoring systems are described in U.S. Pat. Nos. 6,236,317, 6,426,701, 7,819,136, 9,311,802, US 2009/0195385, 2011/0057799A1, US 2015/0199883, WO 2003/082351, WO 2014/4075456 and WO2016172390. However, these systems tend to be bulky or complicated, some using technologies such as active RFID or similar which require managing a large inventory of user ID badges, for example, amongst other deficiencies.

This background information is provided to reveal information believed by the applicant to be of possible relevance. No admission is necessarily intended, nor should be construed, that any of the preceding information constitutes prior art or forms part of the general common knowledge in the relevant art.

### SUMMARY

The following presents a simplified summary of the general inventive concept(s) described herein to provide a basic understanding of some aspects of the disclosure. This summary is not an extensive overview of the disclosure. It is not intended to restrict key or critical elements of embodiments of the disclosure or to delineate their scope beyond that which is explicitly or implicitly described by the following description and claims.

In accordance with one aspect, there is provided a standalone hand wash monitoring device to be mounted in a vicinity of a corresponding sink to monitor, for a given user, a designated hand washing sequence of designated hand washing activities comprising at least a sink-based washing activity and an external washing activity in accordance with respective prescribed washing activity durations, the device comprising: a sensor operable to both detect a user presence of the given user in the vicinity of the sink and further distinguishingly detect a hand sink presence within the sink to distinguishingly detect performance of the sink-based washing activity from performance of the external washing activity; an indicator operable to respectively instruct performance of the designated hand washing activities to the user in accordance with the designated sequence; and a digital timer operatively linked to said sensor and said indicator, and operable to distinguishingly time said user presence and said hand presence against the respective prescribed washing activity durations for compliance with the designated sequence, and activate said indicator accordingly.

In some embodiments, the sensor comprises a user vicinity sensor operable to detect said user presence of the given user in the vicinity of the sink, and a distinct hand sensor operable to distinguishingly detect said hand presence.

In some embodiments, the user vicinity sensor comprises a first point sensor, and wherein said hand sensor comprises a second point sensor.

In some embodiments, the hand sensor comprises a heat sensor.

In some embodiments, the sensor comprises a depth map sensor to distinguishingly detect said user vicinity and said hand presence.

In some embodiments, the device further comprises one of a microcontroller or a microprocessor for operating said digital timer.

In some embodiments, the device further comprises a digital processor for processing a signal received from said sensor and apply one or more detection confirmation confidence thresholds thereto in confirming one or more of said a user presence or said hand presence.

In some embodiments, the hand sensor hand sensor comprises a physically adjustable sensor so to be physically adjusted once the device is mounted in the vicinity of the sink to orient said hand sensor to detect said hand presence within the sink.

In some embodiments, the one or more indicators comprise one or more backlit indicator icons, wherein each of said icons illustrate graphically each of the designated hand washing activities.

In some embodiments, the sink-based washing activity comprises a hand rinsing activity, and wherein the external washing activity comprises a scrubbing activity.

In some embodiments, the device further comprises a communication interface to a soap dispenser comprising a corresponding soap dispenser interface and a soap dispensing sensor operable to detect a hand soap dispensing activity, the device further operable to process said hand soap dispensing activity for compliance with the designated sequence.

In some embodiments, the hand soap dispensing activity comprises at least one of a soap dispensing activity or a hand soap dispenser presence at said soap dispenser.

In some embodiments, the external hand washing activity is operatively confirmed upon detecting the user presence in absence of detecting the hand sink presence according to the designated sequence.

In accordance with another aspect, there is provided a hand wash monitoring system to monitor, for a given user, a designated hand washing sequence of designated hand washing activities comprising at least a sink-based washing activity and an external washing activity in accordance with respective prescribed washing activity durations, the system comprising: a sensor to be mounted in a vicinity of a corresponding sink and operable to both detect a user presence of the given user in the vicinity of the sink and further distinguishingly detect a hand sink presence within the sink to distinguishingly detect performance of the sink-based washing activity from performance of the external washing activity; an indicator operable to respectively instruct performance of the designated hand washing activities to the user in accordance with the designated sequence; and a digital timer operatively linked to said sensor and said indicator, and operable to distinguishingly time said user presence and said hand presence against the respective prescribed washing activity durations for compliance with the designated sequence, and activate said indicator accordingly.

In some embodiments, the sensor comprises a user vicinity sensor operable to detect said user presence of the given user in the vicinity of the sink, and a distinct hand sensor operable to distinguishingly detect said hand presence.

In some embodiments, the hand sensor comprises a physically adjustable sensor so to be physically adjusted once the device is mounted in the vicinity of the sink to orient said hand sensor to detect said hand presence within the sink.

In some embodiments, the sink-based washing activity comprises a hand rinsing activity, and wherein the external washing activity comprises a scrubbing activity.

In some embodiments, the system further comprises a soap dispensing sensor operable to detect a hand soap dispensing activity, wherein the device is further operable to process said hand soap dispensing activity for compliance with the designated sequence.

In some embodiments, the hand soap dispensing activity comprises at least one of a soap dispensing activity or a hand soap dispenser presence at a soap dispenser.

In some embodiments, the external hand washing activity is operatively confirmed upon detecting the user presence in absence of detecting the hand sink presence according to the designated sequence.

In some embodiments, the system further comprises an entry monitoring sensor operatively linked to an entry door leading to said designated sink.

Other aspects, features and/or advantages will become more apparent upon reading of the following non-restrictive description of specific embodiments thereof, given by way of example only with reference to the accompanying drawings.

### BRIEF DESCRIPTION OF THE FIGURES

Several embodiments of the present disclosure will be provided, by way of examples only, with reference to the appended drawings, wherein:

FIG. 1 is a schematic diagram of an exemplary hand washing monitoring system, in accordance with one embodiment;

FIG. 2 is a perspective view of an exterior enclosure of a hand washing monitoring device, including a plurality of backlit icons representing hand washing steps, in accordance with one embodiment;

FIG. 3 is a process flow diagram of an illustrative process for assessing user compliance with a set of visually communicated hand washing steps, in accordance with one embodiment;

FIGS. 4A to 4D are process flow diagrams of an illustrative process for assessing user compliance with a set of visually communicated hand washing steps, in accordance with another embodiment;

FIGS. 5A and 5B are process flow diagrams used to confirm the presence of a user or a user's hands, respectively, using the user vicinity sensor and hand sensor of FIG. 1, in accordance with one embodiment;

FIG. 6 is a process flow diagram of the D4 delay function of FIG. 5C, in accordance with one embodiment; and

FIGS. 7A to 7C are hardware schematics of exemplary interconnections between a power management system, microcontroller and peripherals, respectively, of a hand washing, in accordance with one embodiment.

Elements in the several figures are illustrated for simplicity and clarity and have not necessarily been drawn to scale. For example, the dimensions of some of the elements in the figures may be emphasized relative to other elements for facilitating understanding of the various presently disclosed embodiments. Also, common, but well-understood elements that are useful or necessary in commercially feasible embodiments are often not depicted in order to facilitate a less obstructed view of these various embodiments of the present disclosure.

### DETAILED DESCRIPTION

Various implementations and aspects of the specification will be described with reference to details discussed below. The following description and drawings are illustrative of the specification and are not to be construed as limiting the specification. Numerous specific details are described to provide a thorough understanding of various implementations of the present specification. However, in certain instances, well-known or conventional details are not described in order to provide a concise discussion of implementations of the present specification.

Various apparatuses and processes will be described below to provide examples of implementations of the system disclosed herein. No implementation described below limits any claimed implementation and any claimed implementations may cover processes or apparatuses that differ from those described below. The claimed implementations are not limited to apparatuses or processes having all of the features of any one apparatus or process described below or to features common to multiple or all of the apparatuses or processes described below. It is possible that an apparatus or process described below is not an implementation of any claimed subject matter.

Furthermore, numerous specific details are set forth in order to provide a thorough understanding of the implementations described herein. However, it will be understood by those skilled in the relevant arts that the implementations described herein may be practiced without these specific details. In other instances, well-known methods, procedures and components have not been described in detail so as not to obscure the implementations described herein.

In this specification, elements may be described as “configured to” perform one or more functions or “configured for” such functions. In general, an element that is configured to perform or configured for performing a function is enabled to perform the function, or is suitable for performing the function, or is adapted to perform the function, or is operable to perform the function, or is otherwise capable of performing the function.

It is understood that for the purpose of this specification, language of “at least one of X, Y, and Z” and “one or more of X, Y and Z” may be construed as X only, Y only, Z only, or any combination of two or more items X, Y, and Z (e.g., XYZ, XY, YZ, ZZ, and the like). Similar logic may be applied for two or more items in any occurrence of “at least one . . . ” and “one or more . . . ” language.

The systems and methods described herein provide, in accordance with different embodiments, different examples in which a standalone hand wash monitoring device is mounted in a vicinity of a corresponding sink to, for a given user, monitor a designated hand washing sequence of designated hand washing activities or steps. The device described herein, according to some embodiments, uses a combination of sensors, indicators and timers to provide instructions to the user and monitor the user's compliance with each hand washing step or activity.

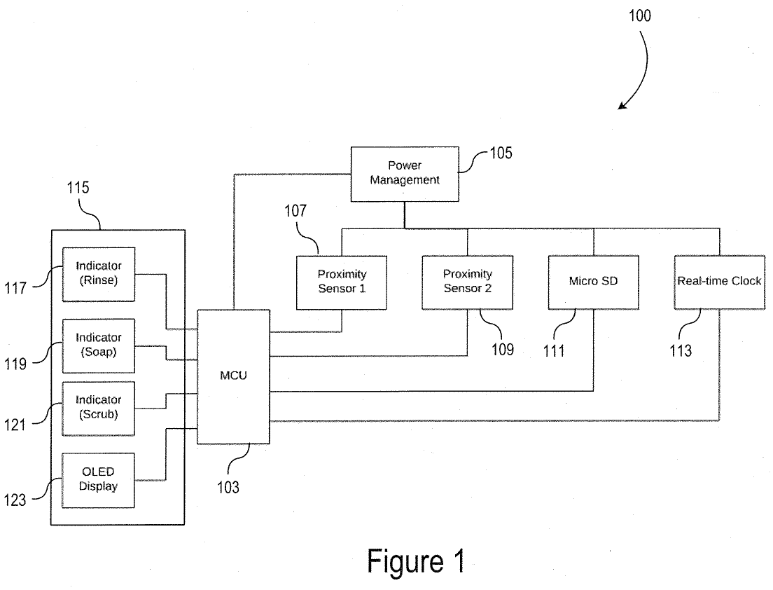

With reference to FIG. 1, and in accordance with one exemplary embodiment, a standalone hand washing monitoring device to be mounted in the vicinity of a corresponding sink, generally referred to using the numeral 100, will now be described. The device generally comprises an internal printed circuit board, which itself comprises a microcontroller unit (MCU) 103 and a power management system (PMS) 105. Different examples of MCU hardware solutions may be used. For example, in some embodiments, an Arduino Pro Mini MCU or similar solution may be used. Other embodiments may instead rely on a microprocessor or the like, for example, an ARM microprocessor or the like, to provide digital control resources for the device 100. Various digital processing, storage and like resources may be included in this respect, as will be readily appreciated by the skilled artisan, to control and govern operation of the device 100, as further detailed below. The power management system 105 is operable to provide power to MCU 103 and other components described below. In some embodiments, the power management system 105 comprises an internal battery and does not require to be operationally connected to an external power source.

In the illustrated embodiment, a set of device components are operationally connected to both the MCU 103 and power management system 105 to implement the herein described hand washing monitoring process. For example, in the illustrated embodiment the device 100 comprises distinct proximity sensors, in this case consisting of a user vicinity sensor 107 and a distinct hand sensor 109, the former operable to detect a user's presence at or vicinity to the device, and thus the sink by which the device is installed, while the latter is operable to distinguishingly detect a presence of the user's hands in the sink. In some embodiments, the user vicinity sensor 107 and/or hand sensor 109 may comprise infrared proximity sensors such as Lidar proximity sensors or the like, though other distinct point sensor technologies may also be considered without departing from the general scope and nature of the present disclosure. In other embodiments, a common sensor, such as a 3D depth map sensor or the like (e.g. similar to that implemented by the Microsoft Kinect™ device) may be operated to detect both the user and the user's hand presence near and within the sink, respectively. For simplicity of description, the following will refer to distinct vicinity and hand sensors to encompass both the possibility of distinct hardware sensors and a common sensor technology capable of computationally distinguishing sensor zones.

Generally, the field of detection of the user vicinity sensor 107 will encompass the general vicinity in front of the sink around which the device has been installed (e.g. to monitor for external hand washing activities such as soaping and scrubbing), whereas the field of detection of the hand sensor 109 will generally encompass an area within or above the sink so to be used to distinguishingly monitor the presence of the user's hands inside the sink (e.g. to distinguishingly monitor for sink-based washing activities such as wetting or rinsing). Therefore, the hand sensor's general detection orientation should be different from that of the user vicinity sensor 107 and should generally be limited to a detection zone within and/or above the sink. In this respect, and in accordance with some embodiments, the hand sensor 109 may be installed onto a rotation mount or removable module to allow for easy adjustment of the hand sensor's orientation angle. For single sensor implementations, distinct vicinity and hand sensor zones may otherwise be dynamically adjusted computationally. Such adjustability may be particularly advantageous to accommodate different device installations where dimensions/configurations of the walls/counter/sink in different scenarios may be unknown prior to installation and/or changed over time, thus allowing for orientation of the hand sensor 109 to be optimized during/after installation.

As illustrated in the exemplary embodiment of FIG. 1, the device 100 may further comprise a digital storage device 111, such as a removable storage device (e.g. removable media/SD card reader/writer), a wired or wirelessly accessible digital storage device (e.g. Wi-Fi, Bluetooth™, NFC, USB accessible storage device), or the like to store hand wash monitoring data for downstream processing, for example, in compiling hand washing compliance metrics, statistics and analyses. A digital timer clock 113 is also included in the illustrated embodiment so to provide visual countdown features in operation.

With continued reference to FIG. 1, the device 100 further comprises an indicator panel 115 operable to instruct performance of a designated hand washing activities to the user in accordance with a designated sequence. In the exemplary embodiment of FIG. 1, panel 115 comprises a plurality of backlit indicator icons, each icon indicative of a corresponding hand washing step. The illumination of each icon is controlled via a LED or similar, operatively connected to MCU 103. In some embodiments, an e-ink (electronic ink) display or similar may be used instead to replicate the indicator icons. In the currently discussed exemplary embodiment, three icons are used: rinse indicator icon 117, soap indicator icon 119 and scrub indicator icon 121. In some embodiments, panel 115 may further comprise a LED or OLED digital display to provide additional information, such as for example a time counter or similar. Other types of indicator devices may be used, for example auditory indicator devices such as speakers for providing sound cues (i.e. buzzer) and/or verbal instructions.

In some embodiments, based on sensor information and an exemplary algorithm described below, the MCU is operable to determine the stage of hand washing of a user and illuminate the correct indicator icons along with a countdown timer via digital clock 113.

In some embodiments, additional modules and/or accessories may be provided to add new features to system 100. For example, in some embodiments, a soap dispenser module, comprising an IR sensor for detecting the presence of a hand obtaining soap, or again comprising a soap dispensing sensor for detecting a soap dispensing action, may be attached or otherwise mounted in relation to, or integrated with, a soap dispenser and operationally (wired or wirelessly) connected to system 100. Such a soap dispenser module may be operable to record soap dispensing events (e.g. with a timestamp), which can be synchronized with the other devices of system 100. In another example, an entrance monitor may be used to detect the opening or closing of doors to monitor inflow and outflow of users and record data with a synchronized timestamp as well. Such an entrance monitor may use a magnetic switch system or similar, for example.

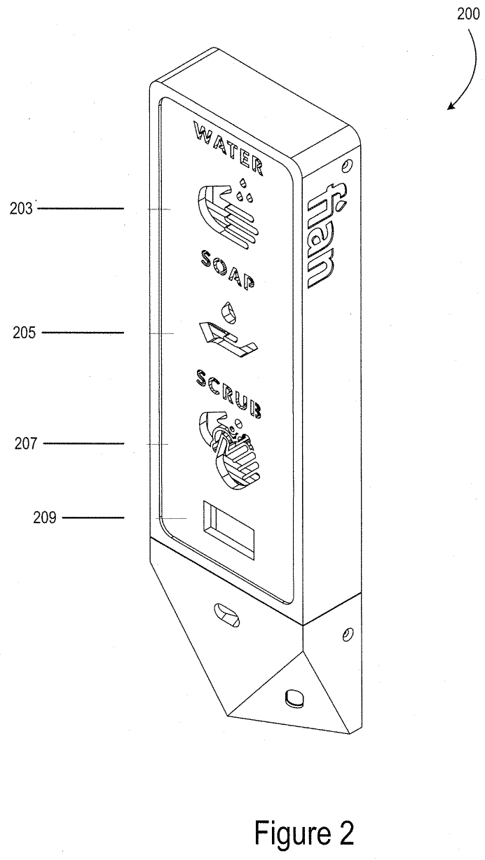

With reference to FIG. 2, and in accordance with one embodiment, a perspective view of an exemplary hand washing monitoring device, generally referred to using numeral 200, will be described. FIG. 2 illustrates an exemplary casing comprising a front panel with a plurality of backlit indicator icons. In this exemplary embodiment, the panel comprises backlit illustrations or icons corresponding to a sequence of hand washing steps, including a water indicator icon 203, a soap indicator icon 205, and a scrub indicator icon 207. Below the icons is located an aperture for OLED digital display 209 or similar, as discussed above. The skilled artisan will understand that some indicator icons may be removed or that additional indicator icons may be added, to provide for a more accurate hand washing sequence, without limitation.

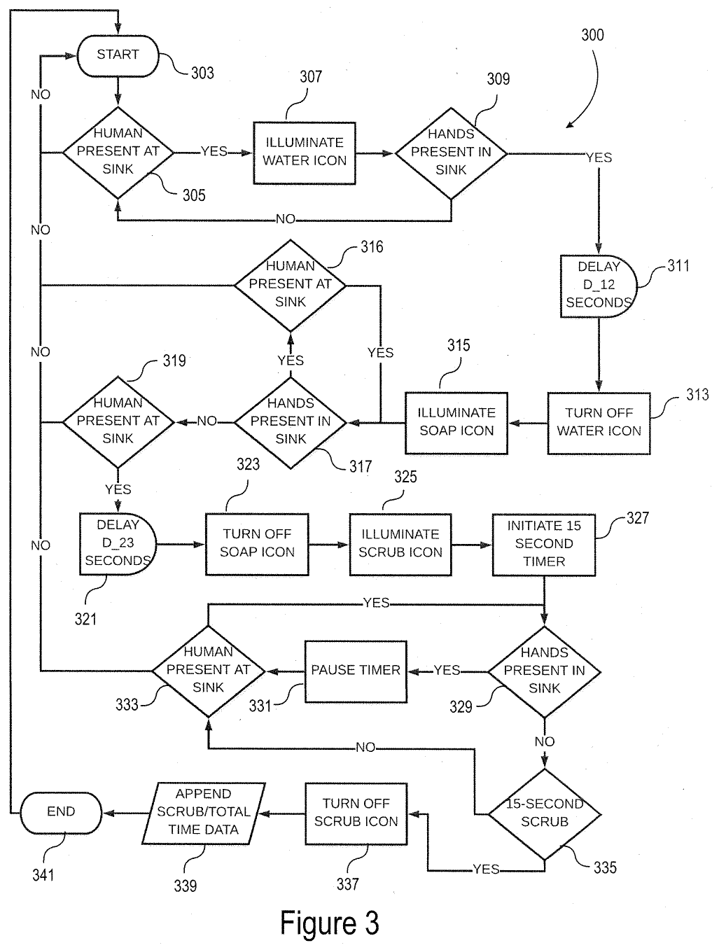

With added reference to FIG. 3, and in accordance with one embodiment, an exemplary process for monitoring or assessing user compliance with a sequence of designated hand washing activities, generally referred to using the numeral 300, will now be described. The method is first initiated at step 303, at which point the hand washing monitoring device is assumed to be correctly installed, for example in front of a sink as described above or similar, and powered on. Upon detection of a human person or user in the general vicinity of the sink at step 305, via user vicinity sensor 107, a water icon indicator 203 is illuminated to instruct the user to first open the tap and wet his/her hands. The method then checks for the presence of the user's hands within the sink region via hand sensor 109. If no confirmation that the user's hands are present in the vicinity of the sink is received, the method reverts back to step 305. In the case where the hands are detected, the method waits for a given amount of time (step 311), here D_12 seconds (e.g. 2 seconds), as to give enough time for the user to thoroughly wet his/her hands. Water indicator icon 203 is then turned off (step 313), followed by illumination of soap indicator icon 205 (step 315) to instruct the user to obtain and apply soap to his/her hands. Once this is done, the method verifies that the user's hands are still present in the sink vicinity via hand sensor 109 (step 317), in which case the soap application step hasn't been followed, therefore prompting the method to go back to step 315. If no hands are detected within the sink, the method ensures that the user himself/herself did not leave the room via user vicinity sensor 107 (step 319). If no user presence is detected, another check is made that the user is still present (step 316), in which case the system goes back to step 315. If the user is not detected then it is assumed that the user has left without completing the hand washing procedure and the system goes back to stand-by at step 303. If the user's hands are not detected but the user is still present, the system waits again (step 321) for an appropriate amount of time (here D_23 seconds), after which soap icon 205 is turned off (step 323) and scrub indicator icon 209 is illuminated (step 325). At this point, a timer is initiated to monitor the scrub time (step 327). To confirm that the user is really scrubbing his/her hands while the timer is activated, at any point during this period, the method may verify (continuously or at fixed time intervals) if the user's hands are present in the sink (step 329), in which case it is assumed that the user has stopped scrubbing and is rinsing instead. Therefore, the system pauses the timer (step 331) and once more verifies that the user himself/herself is still present in the sink's vicinity (step 333). If this isn't the case, the user is once more assumed to have left and the system goes back in stand-by (step 303). If the user is still present but with his/her hands in the sink, the method flashes the scrub indicator icon to remind the user to remove his/her hands form the sink and keep scrubbing. As long as no hands are detected within the sink, the timer elapses until the end of the scrub period (here 15 seconds for example at step 335). Once the scrub period is over, the scrub indicator icon is turned off (step 337). The method finally appends (step 339) the total time of the entire process since the user was first detected and/or scrub time to memory, for example storage device 111. At step 341, the washing sequence is now deemed over and the user may rinse his/her hands and leave the sink. The device then goes back to step 303 and waits for another user. It will be apparent to the skilled technician that different time delays to those illustrated in FIG. 3 may be used. Moreover, in some embodiments, variable time delays may be used depending on additional inputs provided by additional sensor types, as discussed above. The process of FIG. 3 describes a generalized procedure which may be implemented in software to interact with the described hardware in many ways. One of these is described below.

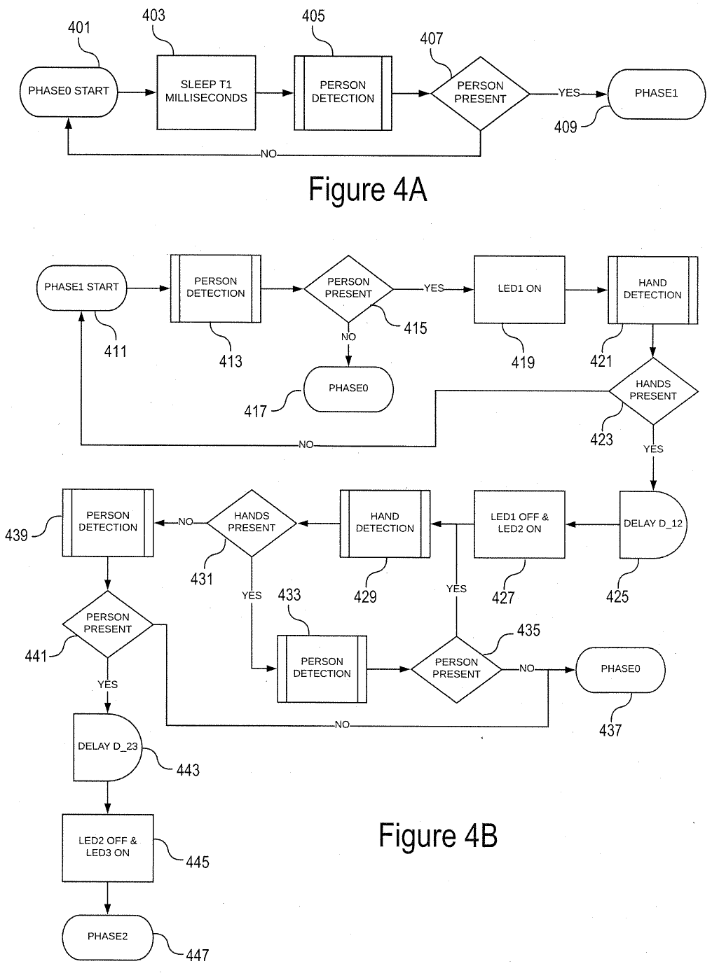

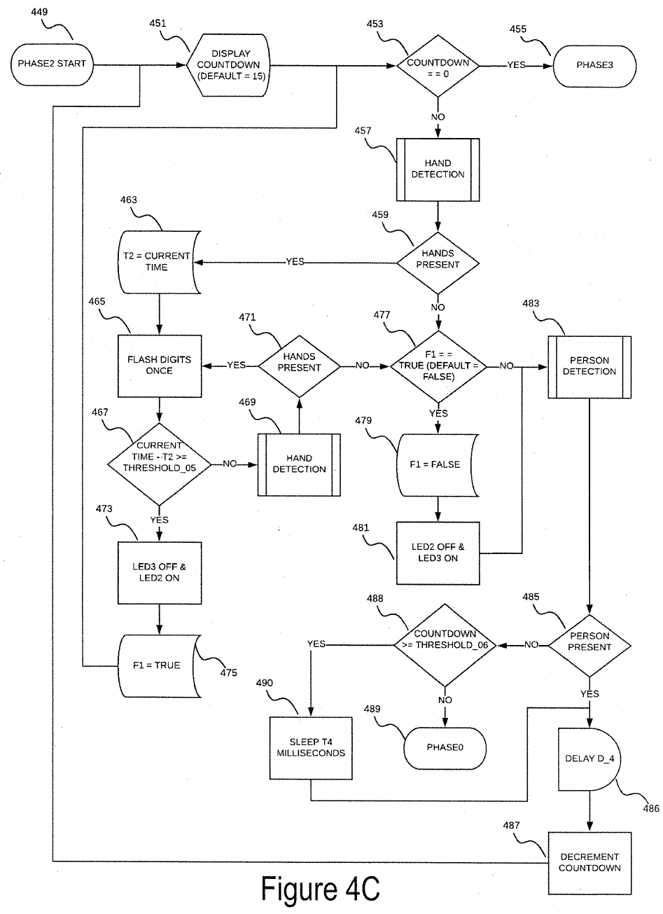

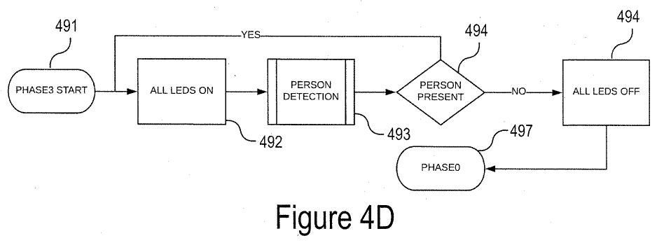

With reference to FIGS. 4A to 4D, and in accordance with one embodiment, a second exemplary process for monitoring or assessing user compliance with a sequence of designated hand washing activities will now be described. The process described below, according to one embodiment, reproduces most of the functionalities of the more general process of FIG. 3, but with the added details with respect to the software implementation of said functionalities using the hardware described above (e.g. exemplary hardware of FIGS. 1 and 2). For added clarity, the process described below is subdivided into 4 sub-processes (phase0, phase1, phase2, and phase3).

Moreover, the array of variables used in the following process described in FIGS. 4A to 4D are listed in the table below with a short description:

TABLE 1

| | Description |
|---|---|
| Thresholds variables | |
| THRESHOLD_01 | Person distance threshold, this value is set automatically during |
| | calibration. It is the basis for deciding a person's presence in |
| | PERSON PRESENT FUNCTION( ) |
| THRESHOLD_02 | Person presence confidence threshold |
| THRESHOLD_03 | Hand distance threshold, this value is set automatically during |
| | calibration. It is the basis for deciding hand presence in HANDS |
| | PRESENT FUNCTION( ) |
| THRESHOLD_04 | Hand presence confidence threshold |
| THRESHOLD_05 | Threshold for how long to wait before triggering soap again |
| THRESHOLD_06 | Threshold to prevent device from switching off if user moves |
| | away from device to get soap from a nearby dispenser |
| THRESHOLD_07 | Threshold to allow for a short delay to pass before decrementing |
| | the countdown timer |
| Sleep Variables | |
| T1 | sleep while no one is there for t1 milliseconds |
| T2 | record time at which the flashing was started if user is not |
| | adhering to scrub phase |
| T3 | Record time at which delay function D_4 was first begun |
| Delay variables | |
| D_12 | Water to Soap Delay |
| D_23 | Soap to Scrub Delay |
| D_4 | Makes sure 1 second passed before decrementing countdown |
| Flag Variable | |
| F1 | Flag that the Soap LED was re-triggered when person is |
| | underneath faucet for too long during scrub phase when OLED is |
| | flashing at him |

| | | Default Value |
|---|---|---|
| Sensor Parameters | Description | (if any) |
| SENSOR 1 | Person Sensor | NA |
| CYCLES_1 | Counter for how many sensor1 readings taken | Default = 0 |
| K1 | Counter for how many readings are within | Default = 0 |
| | threshold_01 | |
| N1 | Number of times to read from sensor1 | NA |
| SENSOR2 | Hand sensor | NA |
| CYCLES_2 | Counter for how many sensor2 readings taken | Default = 0 |
| K2 | Counter for how many readings within | Default = 0 |
| | threshold_03 | |
| N2 | Number of times to read from sensor 2. | NA |

| Other Variables | Description |
|---|---|
| COUNTDOWN | Countdown displayed on OLED screen, |
| | decremented every loop through phase2. |
| CURRENT_TIME | Function that gives you the current time |
| | from the RTC. |

Moreover, in the following description, the different LEDs illuminating the icons are listed as LED1 (rinse icon or indicator 117)), LED2 (soap icon or indicator 119) and LED3 (scrub icon or indicator 121).

Turning to FIG. 4A, the phase0 subprocess, according to one embodiment, will now be described. This subprocess is an exemplary implementation of the device's stand-by mode (step 303 of FIG. 3). In it, the system executes a loop wherein the apparatus or device “sleeps” for T1 milliseconds (step 403) every iteration to conserve power, before trying to detect a user's presence (step 405) via a PERSON PRESENT function (step 407). which will be described in detail below. If no user is detected, the system simply goes back to sleep (step 403) before trying again. In contrast, when a user is detected (step 407), the system then proceeds to phase1 (step 409), as described below. In essence, by changing the value of T1, the rate of detection may be controlled. For example, if only a few users are known to wash their hands every hour (for example), it may be possible to increase T1 (thus reducing the frequency of detection calls) as to conserve battery power.

Now referencing FIG. 4B, the phase1 subprocess will be explained. Once phase1 is started (step 411), the device queries user vicinity sensor (steps 413) and extracts via the PERSON_PRESENT function (step 415) a confirmation that the user is still there and thus confirming the prior detection in phase0 (step 405 above). If the user presence is not confirmed, the process goes back to the start of phase0 (step 417). In contrast, if the user is still present, the water icon (LED1) is activated (step 419). The process then further proceeds to detect that the user's hands are in the vicinity of the sink (step 421). If not, the process goes back to the start of phase1 (step 401). If so, the process applies a delay D_12 of a few seconds (step 425) to give the user time to water his/her hands. Then the water icon LED is turned off and the soap icon LED is turned on (step 427). The user's hands presence in the sink vicinity is then checked one more time (step 429), in which case it may be implied that the user is not putting soap on his/her hands, which triggers another call to the person vicinity sensor (step 433) via the PERSON_PRESENT function (step 435). If the user is present, the process loops back to step 429 while in the opposite case, the process restarts at the beginning of phase0 (step 437). Going back to step 431, if the user's hands are not detected, which may be assumed to be because the user is fetching soap as instructed, the process still checks if the (user's presence is confirmed) (step 439). If not, the process aborts and goes back to the beginning of phase0 (step 437), if so, then a delay of D_23 seconds is applied (step 443) to give the user time to fetch the soap. The process then proceeds to turn off the soap icon LED and turn on the scrub icon LED (step 445) before proceeding to the start of phase2 (step 447).

Turning now to FIG. 4C. the phase2 subprocess will now be described. From the start of phase2 (step 449), the current value of a COUNTDOWN variable, which is meant to illustrate a countdown timer, is shown on the OLED display (step 451). If this is the first time phase2 is initiated, the default starting value is shown (15 in this example but other values may be used). In essence, each time a full pass through phase2 is completed, the COUNTDOWN variable will be decremented (as explained below). Thus, the current value of the COUNTDOWN variable in the current phase2 iteration is checked (step 453). In the case where the value has reached 0, the scrubbing phase is over and the system proceeds to phase3 (step 455). In contrast, if the COUNTDOWN variable still has a non-zero value, the system proceeds with the rest of the phase2 iteration, wherein the hand sensor is used to check if the user's hands are present (step 457). This means that the user is not scrubbing his/her hands as instructed. Thus, if the HANDS_PRESENT function (step 459) returns that the user's hands are still there, the current time is recorded in a variable called T2 (step 463). The device then proceeds with flashing the digits of the COUNTDOWN variable once (step 465) as a reminder to the user to scrub his/her hands until the COUNTDOWN timer is completely elapsed. In some embodiments, instead of or in addition to flashing the digits, an audio cue like a buzzer sound or similar may be used instead. A loop is then initiated wherein the elapsed time (e.g. current time−T2) is checked. If this elapsed time is lower than a threshold value THRESHOLD_05, then the hand sensor is called (step 469) via the HANDS_PRESENT function (step 471). If the user's hands are not detected, the process proceeds to step 477 (described below), in contrast, as long as the user's hands are still detected, the process goes back to step 467. In the event that the user doesn't remove his/her hands, the elapsed time will eventually reach a value larger than the THRESHOLD_05 at step 467. At this point, the user had his/her hands for too long under in the sink vicinity, resulting in the soap being washed off. Hence, the scrub icon LED is turned off and the soap icon is lit once again (step 476) and a F1 flag variable describing this condition (i.e. soap LED was re-triggered) is set as true (step 475). The device then goes back to step 453.

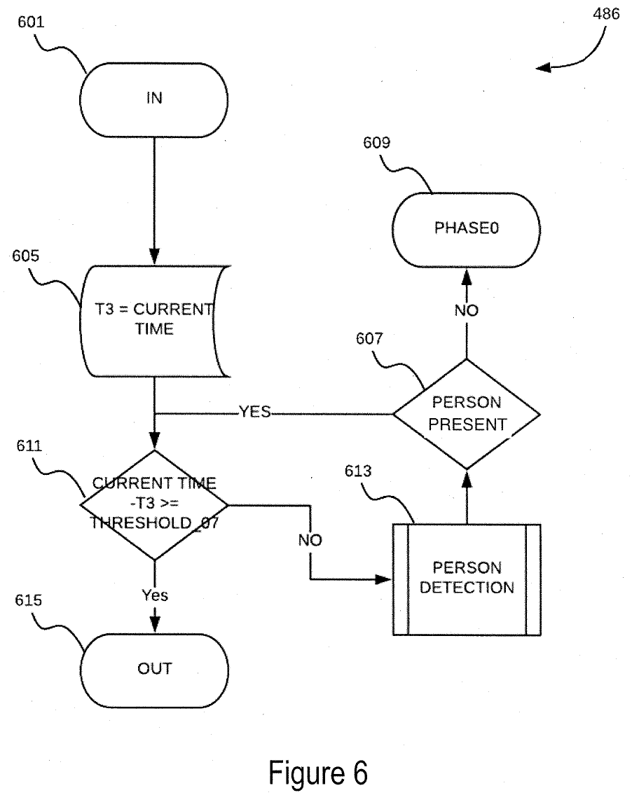

Going back to step the initial detection of the user's hands (step 459), in the case where the user's hands are not detected (which implies that the user is scrubbing his/her hands as instructed), a check is made that the F1 flag variable is not true (step 477). If this is not the case (step 479), due to a previous run through step 475 described above, the soap icon LED is turned off and the scrub icon LED is turned back on (step 481). If the F1 condition is false, the user vicinity sensor function is called to determine that the user is still there (step 483). If the PERSON_PRESENT function (step 485) does confirm that the user is still there (and presumably scrubbing his/her hands), the device activates the delay function D_4 (step 486). As will be described in more detail below with respect to FIG. 6, D4 generally delays the process for a short period of time while also checking that the user is still there. In some embodiments, this period of time is equal to 1 second (meaning that roughly one second will have elapsed each time a phase2 iteration will have occurred, assuming the user respected the instructions, for a total time of roughly 15 seconds for the default value of COUNTDOWN). Once the value of the COUNTDOWN counter variable is decremented (step 487), a new phase2 loop is initiated at step 451. In the case where the user presence is not detected at step 485, the current value of COUNTDOWN is compared to a threshold value of THRESHOLD_06 value. This is done in case (MORE). If the value of COUNTDOWN is smaller than THRESHOLD_06, the system reverts to the standby mode of phase0 (step 493). In contrast, if the value is equal or larger, the device sleeps for T4 milliseconds (steps 495) before proceeding to step 487 described above.

FIG. 4D describes the final subprocess, herein referred to as phase3. This subprocess is called at step 455 of phase2 once the COUNTDOWN reaches zero, implying that the user has scrubbed his/her hands for the prescribed time. This phase is done so as to distinguish the previous user that has just washed his/her hands from a new user. Once phase3 is initiated (step 491), all LEDs are turned on (step 492) to communicate that the scrubbing phase is over to the previous user. The person vicinity sensor is used to check that the user is there (via the PERSON_PRESENT function at step 494). If a user is detected, it is inferred that this is the same user. The process loops back to step 492 to again signal to this user that he/she is done. Thus, the LEDs will flash as long as the user is still present (presumably rinsing his/her hands). Once the user has left and is no longer detected, all LEDs are turned off (step 494) and the device reverts to the stand-by mode of phase0 (step 497), waiting for the next user.

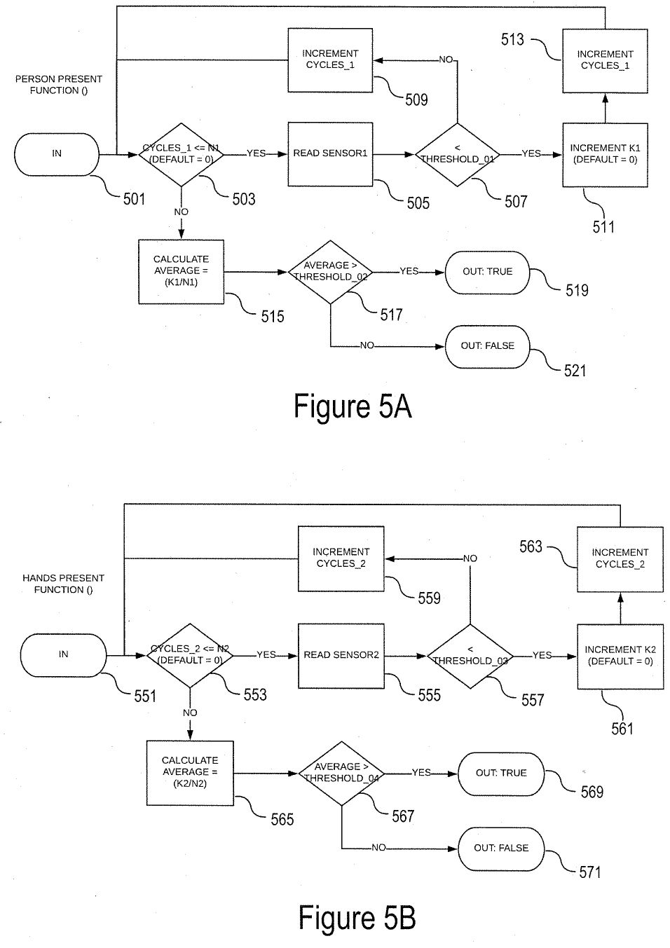

With respect to FIGS. 5A and 5B, the PERSON PRESENT and HANDS PRESENT functions of FIGS. 4A to 4D, respectively, will now be described. The PERSON PRESENT function illustrated in FIG. 5A leverages the user vicinity sensor hardware to reliably identify the presence of a user in the sink vicinity. To avoid false positives, a N1 variable describes the total number of readings to be made with the user vicinity sensor (herein referred to as SENSOR1). After the function is called for the first time (step 501), a check is made (step 503) to verify if a CYCLE_1 counter variable (first initiated to a value of 0) is lower or equal to the total required number of cycles N1. Every time a read cycle is initiated, the system receives the input value from SENSOR1 (step 505), after which it verifies that the input value has a value within a given threshold value TRESHOLD_01 (step 507). This threshold value is determined during calibration. If the input value is too high, another cycle is initiated: the CYCLES_1 variable is incremented by 1 and the process goes back to step 503. In contrast, if the input value is within THRESHOLD_01, both K1, a counter variable indicating the number of times a positive person detection has occurred (from an initial value of zero when the function is called) and CYCLE_1 are incremented (steps 511 and 513). Once the process has cycled through N1 times, it then calculates the percentage of times (step 515), out of the N1 measurements, that the readings were within THRESHOLD_01 (e.g. K1/N1). If that percentage is above a THRESHOLD_02 value (step 517), then the function outputs TRUE (e.g. a person was detected), otherwise it outputs FALSE (e.g. no detection was made) (steps 519 and 521 respectively).

Similarly, FIG. 5B illustrates the HANDS PRESENT function of FIGS. 4A to 4D. This function leverages the hand sensor hardware to reliably identify the presence of the user's hands in the sink vicinity. To avoid false positives, a N2 number of readings are made with the hand sensor (herein referred to as SENSOR2). After the function is called for the first time (step 551), a check is made (step 553) to verify if a CYCLE_2 counter variable (first initiated to a value of 0) is lower or equal to the total required number of cycles N2. Every time a read cycle is initiated, the system receives the input value from SENSOR2 (step 555), after which it verifies that the sensor value has a value within a given threshold value TRESHOLD_03 (step 557). This threshold value is determined during calibration. If the sensor value is too high, another cycle is initiated: the CYCLES_2 variable is incremented by 1 and the process goes back to step 553. In contrast, if the input value is within THRESHOLD_03, both K2, a counter variable indicating the number of times a positive person detection has occurred (from an initial value of zero when the function is called) and CYCLE_2 are incremented. Once the process has cycled through N2 times, it then calculates the percentage of times (step 565), out of the N1 measurements, that the readings were within THRESHOLD_02 (e.g. K1/N1). If that percentage is above a THRESHOLD_04 value (step 567), then the function outputs TRUE (e.g. hands were detected), otherwise it outputs FALSE (e.g. no detection was made) (steps 569 and 571 respectively).

The skilled artisan will understand the thresholds discussed above may be determined during a calibration process to ensure optimal detection reliability and sensitivity. Similarly, optimal values of N1 and N2 may also be determined empirically to optimize reliability while minimizing battery use.

With respect to FIG. 6, the delay function D_4 of step 486 of FIG. 4C will now be described. As mentioned above, this function is designed so that the device waits for a time delay value of THRESHOLD_07 before continuing to the next step while at the same time checking to see if the user is still present or not. Once the function D_4 is initiated (step 601), time is recorded and saved into a variable called T3 (step 605) for future reference. A loop is then initiated, at which point a check is made (step 611) upon the current time to see if a time delay of THRESHOLD_07 has elapsed since entering the function (e.g. the current time is equal or higher than the value of T3+THRESHOLD_07). As mentioned above, in some embodiments this value is equal to 1 second, although other values may be used. If this is case the process stops recording time and leaves the D_4 function (step 615). However, if that time delay is not yet reached, the device instead calls upon the user vicinity sensor (step 613) to determine if the user is still present (step 483). If the PERSON_PRESENT function described above (step 607) cannot detect the user, then it is assumed that the user left and the device goes back into stand-by mode phase0 (step 609). If the user is still there, the device loops back to step 605 and checks once more to see if the elapse time since entering the D_4 function is equal or higher than a value of THRESHOLD_07.

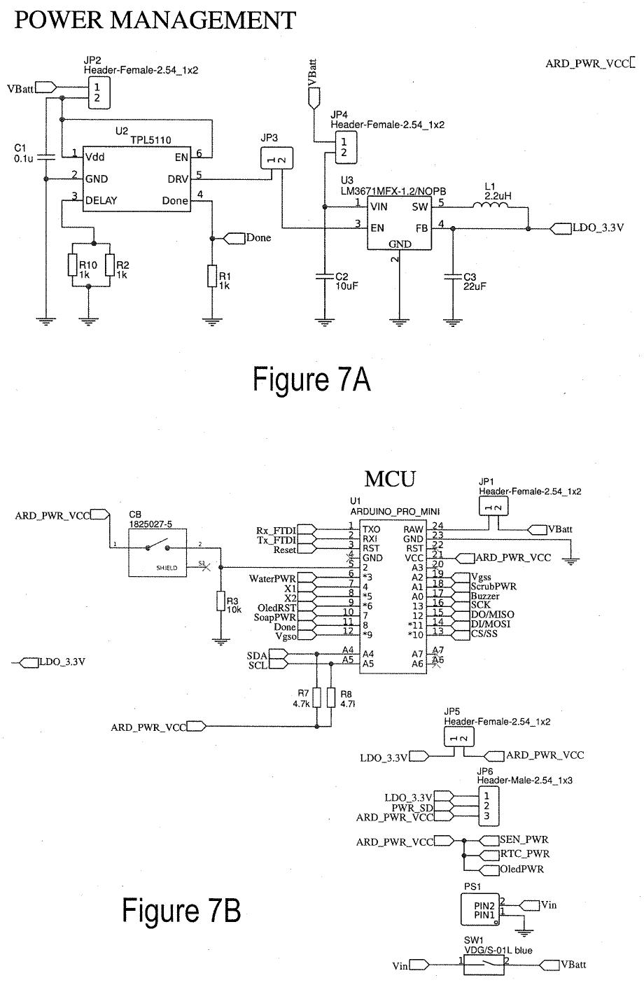

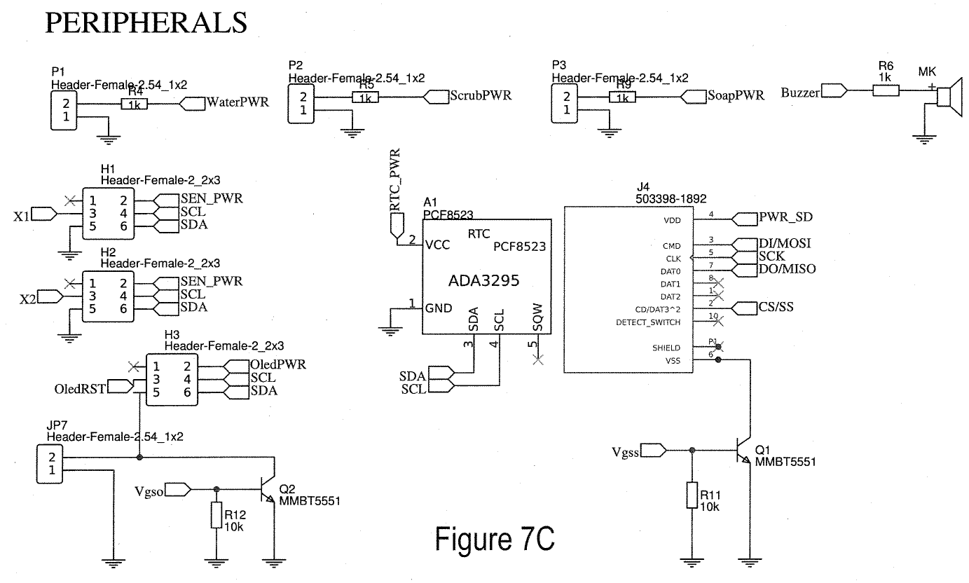

FIGS. 7A to 7C illustrate hardware schematics of interconnections between a power management subsystem, microcontroller and peripherals of a hand washing monitoring system, such as that described in FIG. 1, according to one embodiment. The skilled artisan will understand that different choices in hardware implementation may be made, without deviating from the general scope and nature of the herein-described embodiments.

While the present disclosure describes various embodiments for illustrative purposes, such description is not intended to be limited to such embodiments. On the contrary, the applicant's teachings described and illustrated herein encompass various alternatives, modifications, and equivalents, without departing from the embodiments, the general scope of which is defined in the appended claims. Except to the extent necessary or inherent in the processes themselves, no particular order to steps or stages of methods or processes described in this disclosure is intended or implied. In many cases the order of process steps may be varied without changing the purpose, effect, or import of the methods described.

Information as herein shown and described in detail is fully capable of attaining the above-described object of the present disclosure, the presently preferred embodiment of the present disclosure, and is, thus, representative of the subject matter which is broadly contemplated by the present disclosure. The scope of the present disclosure fully encompasses other embodiments which may become apparent to those skilled in the art, and is to be limited, accordingly, by nothing other than the appended claims, wherein any reference to an element being made in the singular is not intended to mean “one and only one” unless explicitly so stated, but rather “one or more.” All structural and functional equivalents to the elements of the above-described preferred embodiment and additional embodiments as regarded by those of ordinary skill in the art are hereby expressly incorporated by reference and are intended to be encompassed by the present claims. Moreover, no requirement exists for a system or method to address each and every problem sought to be resolved by the present disclosure, for such to be encompassed by the present claims. Furthermore, no element, component, or method step in the present disclosure is intended to be dedicated to the public regardless of whether the element, component, or method step is explicitly recited in the claims. However, that various changes and modifications in form, material, work-piece, and fabrication material detail may be made, without departing from the spirit and scope of the present disclosure, as set forth in the appended claims, as may be apparent to those of ordinary skill in the art, are also encompassed by the disclosure.

What is claimed is:

1. A standalone hand wash monitoring device to be mounted in a vicinity of a corresponding sink to monitor, for a given user, a designated hand washing sequence of designated hand washing activities comprising at least a sink-based washing activity and an external washing activity in accordance with respective prescribed washing activity durations, the device comprising:
a sensor operable to both detect a user presence of the given user in the vicinity of the sink and further distinguishingly detect a hand sink presence within the sink to distinguishingly detect performance of the sink-based washing activity from performance of the external washing activity;
an indicator operable to respectively instruct performance of the designated hand washing activities to the user in accordance with the designated sequence; and
a digital timer operatively linked to said sensor and said indicator, and operable to distinguishingly time said user presence and said hand presence against the respective prescribed washing activity durations for compliance with the designated sequence, and activate said indicator accordingly.

2. The device of claim 1, wherein said sensor comprises a user vicinity sensor operable to detect said user presence of the given user in the vicinity of the sink, and a distinct hand sensor operable to distinguishingly detect said hand presence.

3. The device of claim 2, wherein said user vicinity sensor comprises a first point sensor, and wherein said hand sensor comprises a second point sensor.

4. The device of claim 2, wherein said hand sensor comprises a heat sensor.

5. The device of claim 1, wherein said sensor comprises a depth map sensor to distinguishingly detect said user vicinity and said hand presence.

6. The device of claim 1, further comprising one of a microcontroller or a microprocessor for operating said digital timer.

7. The device of claim 1, further comprising a digital processor for processing a signal received from said sensor and apply one or more detection confirmation confidence thresholds thereto in confirming one or more of said a user presence or said hand presence.

8. The device of claim 2, wherein said hand sensor comprises a physically adjustable sensor so to be physically adjusted once the device is mounted in the vicinity of the sink to orient said hand sensor to detect said hand presence within the sink.

9. The device of claim 1, wherein said one or more indicators comprise one or more backlit indicator icons, wherein each of said icons illustrate graphically each of the designated hand washing activities.

10. The device of claim 1, wherein the sink-based washing activity comprises a hand rinsing activity, and wherein the external washing activity comprises a scrubbing activity.

11. The device of claim 1, further comprising a communication interface to a soap dispenser comprising a corresponding soap dispenser interface and a soap dispensing sensor operable to detect a hand soap dispensing activity, the device further operable to process said hand soap dispensing activity for compliance with the designated sequence.

12. The device of claim 11, wherein said hand soap dispensing activity comprises at least one of a soap dispensing activity or a hand soap dispenser presence at said soap dispenser.

13. The device of claim 1, wherein the external hand washing activity is operatively confirmed upon detecting the user presence in absence of detecting the hand sink presence according to the designated sequence.

14. A hand wash monitoring system to monitor, for a given user, a designated hand washing sequence of designated hand washing activities comprising at least a sink-based washing activity and an external washing activity in accordance with respective prescribed washing activity durations, the system comprising:
a sensor to be mounted in a vicinity of a corresponding sink and operable to both detect a user presence of the given user in the vicinity of the sink and further distinguishingly detect a hand sink presence within the sink to distinguishingly detect performance of the sink-based washing activity from performance of the external washing activity;
an indicator operable to respectively instruct performance of the designated hand washing activities to the user in accordance with the designated sequence; and
a digital timer operatively linked to said sensor and said indicator, and operable to distinguishingly time said user presence and said hand presence against the respective prescribed washing activity durations for compliance with the designated sequence, and activate said indicator accordingly.

15. The system of claim 14, wherein said sensor comprises a user vicinity sensor operable to detect said user presence of the given user in the vicinity of the sink, and a distinct hand sensor operable to distinguishingly detect said hand presence.

16. The system of claim 15, wherein said hand sensor comprises a physically adjustable sensor so to be physically adjusted once the device is mounted in the vicinity of the sink to orient said hand sensor to detect said hand presence within the sink.

17. The system of claim 14, wherein the sink-based washing activity comprises a hand rinsing activity, and wherein the external washing activity comprises a scrubbing activity.

18. The system of claim 14, further comprising a soap dispensing sensor operable to detect a hand soap dispensing activity, wherein the device is further operable to process said hand soap dispensing activity for compliance with the designated sequence.

19. The system of claim 18, wherein said hand soap dispensing activity comprises at least one of a soap dispensing activity or a hand soap dispenser presence at a soap dispenser.

20. The system of claim 14, wherein the external hand washing activity is operatively confirmed upon detecting the user presence in absence of detecting the hand sink presence according to the designated sequence.

21. The system of claim 14, further comprising an entry monitoring sensor operatively linked to an entry door leading to said designated sink.
